(19) United States
(12) Patent Application Publication
(10) Pub. No.: US 2006/0112630 A1
(43) Pub. Date: Jun. 1, 2006

(54) High efficiency automatic plant cloning system

(76) Inventor: Conrad P. Kimes, Laguna Niguel, CA (US)

Correspondence Address: Conrad P. Kimes;c/o Burge & Strid, 23193 La Cadena Drive, Suite 101, Laguna Hills, CA, 92653, US

(21) Appl. No.: 10/991,108

(22) Filed: Nov. 17, 2004

(51) Int. Cl. A01G 31/00

(52) U.S. Cl. 47/62 C

(57) ABSTRACT

A cost effective high efficiency automatic plant cloning system which provides for accelerated plant shoot propagation is disclosed. A water and nutrient re-circulating system that is controlled off and on provides plant shoot immersion wetting and drying cycles. Plant shoots are supported in porous fibrous plant shoot medium that is placed in water and nutrient conducting channels where the water and nutrient in the on cycle contact and repeatedly wet the plant shoot and its emerging root structure. The porous fibrous plant shoot medium also provides for the growing plant shoot to expand without any hindering of the plant shoot and emerging root structure. The porous fibrous plant shoot support medium also provides for the circulation of air and absorption of light with ultra violet rays for maintaining the porous fibrous plant shoot medium free from adverse growth mechanisms, while at the same time providing for a dark zone in which the plant shoot root structure takes place In the off cycle, air dries the plant shoot and the emerging root structure. The on-off cycle of wetting and drying is controlled automatically to provide optimum and accelerated growth of the plant shoots to developed root structure for early transplanting to a soil environment with a minimal loss of plant shoot material.

### BACKGROUND OF THE INVENTION

1. Field of the Invention

The present invention relates to the botanical sciences and specifically to an apparatus for High Efficiency Automatic Cloning of selected plants that can be reproduced from plant shoots. The present invention is concerned with a practical apparatus where the apparatus provides for efficient means for reproducing plant shoots that can be mounted and exposed alternately to water and nutrient and air in such a controlled manner that healthy root propagation occurs in shortened time periods such that the plants can be transferred to the soil environment in as short as time as possible and in healthy states that promote low per centages of lost plants to the growing process.

2. Description of Related Arts

The ability to successfully propagate plants as identical or near identical plants in efficient manners has long been a goal of plant cultivation. Extensive means of meeting this goal have involved direct soil growing means. For plants capable of rooting from a plant shoot, such a plant shoot as been placed in a soil environment and carefully nurtured to a rooting status and then made available to be planted in more substantial soil environments. In more modern times research has been conducted into cultivation of such plants with water and nutrient solution exposure of the plant shoot without the use of soil in the initial rooting process, such processes being known as hydroponic methods of initial growth without the use of the soil environment.

For the horticultural hobbyist, as well as the small to intermediate grower, sophisticated hydroponic methods for controlling the environment for the rapid growth of plant shoots are beyond the economical capability of such individuals and organizations. There remains a need for cost effective and efficient methods for plant propagation. The following art illustrates the types of means and methods that have developed for use in such plant propagation.

U.S. Pat Application 2003/0051398 A1 provides for the use of a soil substrate useful in supporting plant growth wherein a blend of fibreball natural and synthetic polymer fibers which replace all or most all of a soil base with a biodegradable growth medium.

U.S. Pat. Application 2003/0145579 A1 provides for a closed system for continuous water circulation to promote hydroponic growth.

U.S. Pat. Application 2002/0155595 A1 provides for a hydroponic system for intermittent liquid immersion by a mechanical tipping mechanism that intermittently exposes and brings immersion of the plant.

U.S. Pat. No. 5,597,731 provides for the propagation of plantlets through use of a system in which sealed membrane vessels provide the container for the plant material; the sealed membrane vessels are permeable to gas and liquids to provide the necessary growth materials to the plant.

U.S. Pat. Application 2001/0017004 A1 provides an apparatus for culturing plantlets by photo automatic induced growth, the apparatus comprising elements of a light transmittable and enclosed culture vessel, carbon dioxide and air supply chamber, and fine tubular water supply system.

U.S. Pat. No. 6,000,173 provides for a hydroponic growth station which provides for hydroponic growth of already rooted plants in a pair of growing boards.

U.S. Pat. No. 5,992,093 provides for a designed growth substrate comprised of mineral wool fibers arranged in specified fibre assembly arrangements.

U.S. Pat. No. 5,856,190 provides a multistage plant culture method and apparatus wherein culture beds with rooting medium are contained in culture containers, and the plant growth stages are promoted by movement of the growth stages from one level to another.

U.S. Pat. No. 5,225,342 provides a systemic plant interface by providing a perforate surface with artificial openings or lumens in the micron size range that mates to the growing xylene tissue of a vascular pant and thereby provides fluid communication supply to the plant.

U.S. Pat. No. 5,141,866 provides a washing method and process of plant tissue culture propagation by providing for separating and thinning the plant tissue through a screening in an enclosed environment.

U.S. Pat. No. 5,136,804 provides apparatus for providing water and nutrient through a fog generator which provides a fog mist to the plant.

U.S. Pat. No. 4,926,585 provides for a plant nutrient supply system comprised of a tubular supply system to promote hydroponic growth of the plant, wherein the apparatus provides such nutrients via capillary action through porous members that promote capillary flow of nutrients.

U.S. Pat. No. 4,860,490 provides a hydroponic system wherein a contact pressure system is utilized to promote plant growth.

U.S. Pat. No. 4,793,096 provides a flat handling system for periodic flooding and draining of growing plants and adding and removing plant trays automatically.

U.S. Pat. No. 4,669,217 provides a plant propagation system in an enclosed two tank system which provides periodic flooding of the roots of the propagation plants with plant nutrients.

U.S. Pat. No. 4,302,905 provides for a hydroponic environment growth acceleration by use of a multi medium hydroponic mixture of carbon dioxide gas in a non-miscible liquid medium and use of an aqueous hydroponic nutrient liquid medium; the clone cells are grown in the mixed hydroponic solution.

U.S. Pat. No. 3,365,840 provides a crop growing device for the growth of greenhouse crops, consisting of mechanical arrangements in a configuration to promote ease of handling and growth management.

### SUMMARY OF THE INVENTION

The invention for the High efficiency Automatic Plant Cloning System provides for a water and nutrient supply and return system that provides for an alternating cycle of immersion and drying of plant shoot material. The plant shoot material is mounted in porous fibrous plant shoot support medium that is arranged in alternating water and nutrient conducting channels in a multiple channel extrusion. The porous fibrous plant shoot medium also provides for the growing plant shoot to expand without any hindering of the plant shoot and emerging root structure. The porous fibrous plant shoot support medium also provides for the circulation of air and absorption of light with ultra violet rays for maintaining the porous fibrous plant shoot medium free from adverse growth mechanisms, while at the same time providing for a dark zone in which the plant shoot root structure takes place. The water and nutrient supply to the multiple channel extrusion is provided by a distribution manifold which provides the water and nutrient to the multiple channel extrusion through a plurality of distribution orifices. Aeration of the water and nutrient flow occurs with the water and nutrient flow from the water and nutrient flow conducting channels falling in a waterfall manner back into the water and nutrient supply and receiving resrvoir. The immersion and drying cycles are controlled by an automatic electronic controller. The immersion and drying cycles are continued until a selected root structure is developed for the plant shoot material, wherein the plant shoot and its developed root structure may be placed in a soil environment for complete growth.

### BRIEF DESCRIPTION OF THE DRAWINGS

From the above discussion other objects, characteristics and features and benefits and advantages of the present invention for High Efficiency Automatic Plant Cloning System are made more apparent from consideration of and reference to the following detailed and accompanying drawings, wherein:

FIG. 1 is a schematic illustration showing one example of apparatus for obtaining High Efficiency Automatic Plant Cloning.

FIG. 2 is a schematic representation of a selected plant shoot.

FIG. 3 is a schematic representation of a selected plant shoot placed in a porous fibrous plant shoot support medium.

FIG. 4 is a schematic representation of a selected plant shoot in a porous fibrous plant shoot support medium which has been placed in a water and nutrient transport conducting channel of a multiple channel extrusion.

FIG. 5 is a schematic of a multiple channel extrusion having alternate channels for conducting water flow and alternate surfaces for separating conducting water and nutrient flow channels from each other.

FIG. 6 is a schematic illustration of the multiple channel extrusion being set at an angle with respect to the top of the apparatus in FIG. 1.

FIG. 7 is a schematic illustration of water and nutrient flow through a conducting channel within the extrusion containing multiple channels and containing selected plant shoots mounted in a porous fibrous plant support medium, wherein water and nutrient is flowing down such channel by force of gravity.

FIG. 8 is a schematic illustration of the water and nutrient distributing manifold with a plurality of distributing orifices lined up with the alternating channels of the water and nutrient conducting multiple channel extrusion.

FIG. 9 is a schematic illustration of the water and nutrient distributing manifold with its plurality of distributing orifices.

FIG. 10 is a schematic illustration of a channel within the multiple channel extrusion containing multiple channels and containing selected plant shoots mounted in porous fibrous plant support medium wherein water and nutrient flow has been turned off and air is flowing over the rooting structure of the plant shoots.

### DESCRIPTION OF THE PREFERRED EMBODIMENT

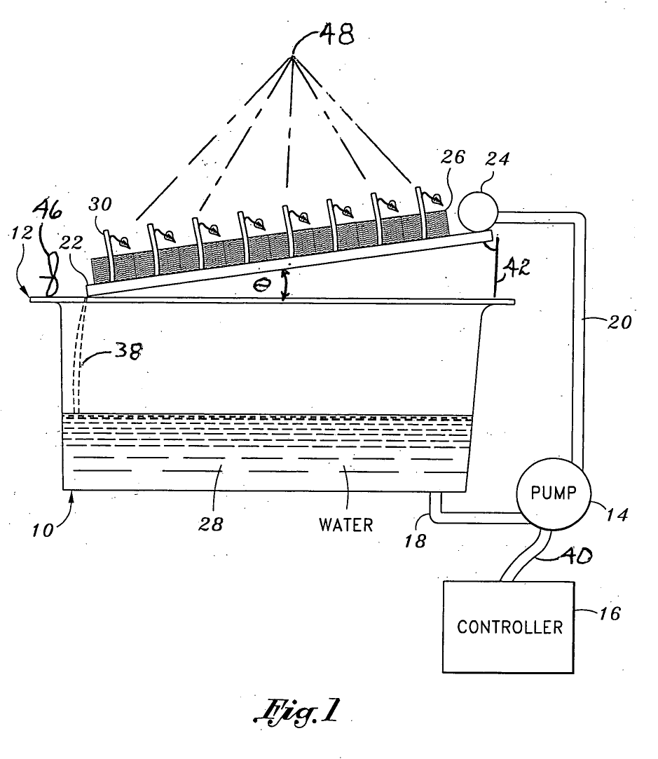

A preferred embodiment of the apparatus for providing economic and high efficient plant cloning for plants from plant shoots comprises as principle elements and arrangements as referenced in the drawings, FIG. 1 through FIG. 10, wherein the characteristics and benefits of the apparatus become more apparent with reference to the makeup and corresponding parts of the apparatus through reference to the drawings.

In FIG. 1 an overall cross-section of the High Efficient Automatic Plant Cloning system apparatus is demonstrated. A water and nutrient supply and return reservoir container 10 with an open surface provides for supplying and returning water and nutrient 28. The water and nutrient supply and return reservoir 10 may be of any suitable shape that is easily manufactured from extrudible or molded plastic. In the water and nutrient 28 supply cycle, such water and nutrient 28 is drawn through a reservoir supply duct 18 to an electrically driven pump 14. The electrically driven pump 14 is cyclically controlled on and off by an electronic controller 16. The electrically driven pump 14 provides sufficient pressure to drive the water and nutrient 28 upward through the pump supply duct 20 to the distributing manifold 24.

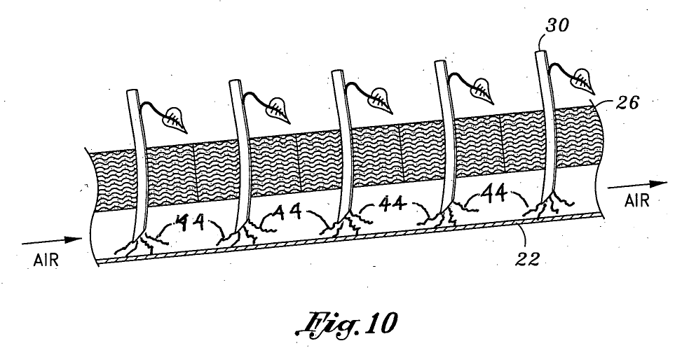

The distributing manifold 24 is provided with a plurality of water and nutrient distributing orifices 32 as shown in FIG. 9. The distributing manifold 24 is arranged and supported at the top of a multiple channel extrusion 22 as shown in FIG. 1. The plurality of water and nutrient distributing orifices 32 are located in reference to the multiple channel extrusion 22 to provide for water and nutrient flowing through such orifices 32 to enter water and nutrient conducting channels 34 and flow downward by the force of gravity through the water and nutrient conducting channels 34. The location of the distributing orifices 32 with respect to the multiple channel extrusion 22 is illustrated in FIG. 8. The return flow of water and nutrient 38 to the water and nutrient supply and return reservoir 10 is located so as to create a waterfall effect which entrains air and provides aeration of the water and nutrient in the water and nutrient supply and return reservoir 10.

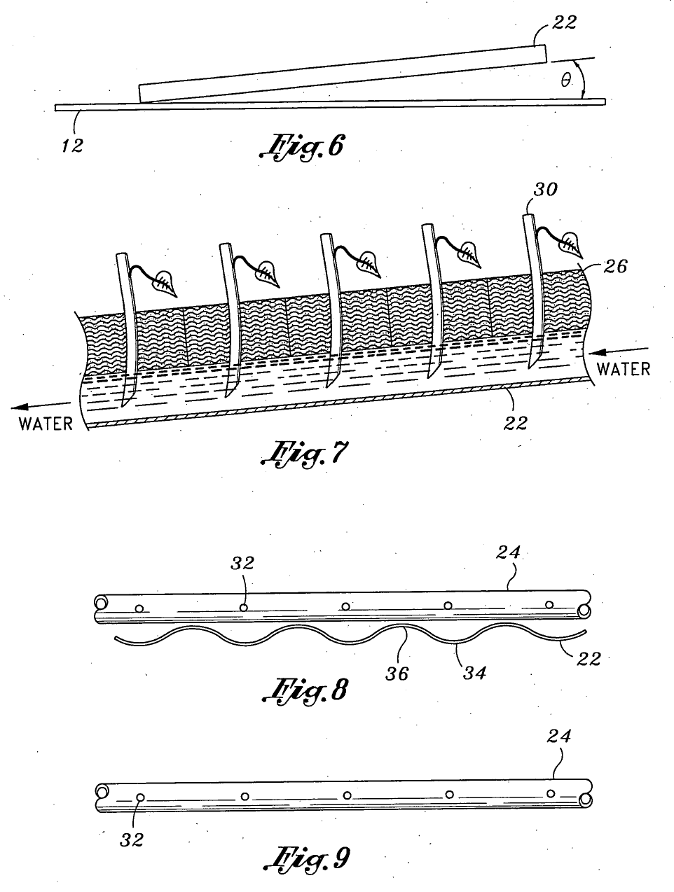

The multiple channel extrusion 22 is placed at an angle θ with respect to a horizontal plane on top of the water and nutrient supply and return reservoir 10, being supported by a supporting porous plate 12 that will not impede the return flow of water and nutrient 38 waterfall to the water and nutrient supply and return reservoir 10. Any suitable support means 42 may be utilized to maintain the angle θ between the multiple channel extrusion 22 and the supporting porous plate 12. The angle θ is illustrated in FIG. 1 and FIG. 6.

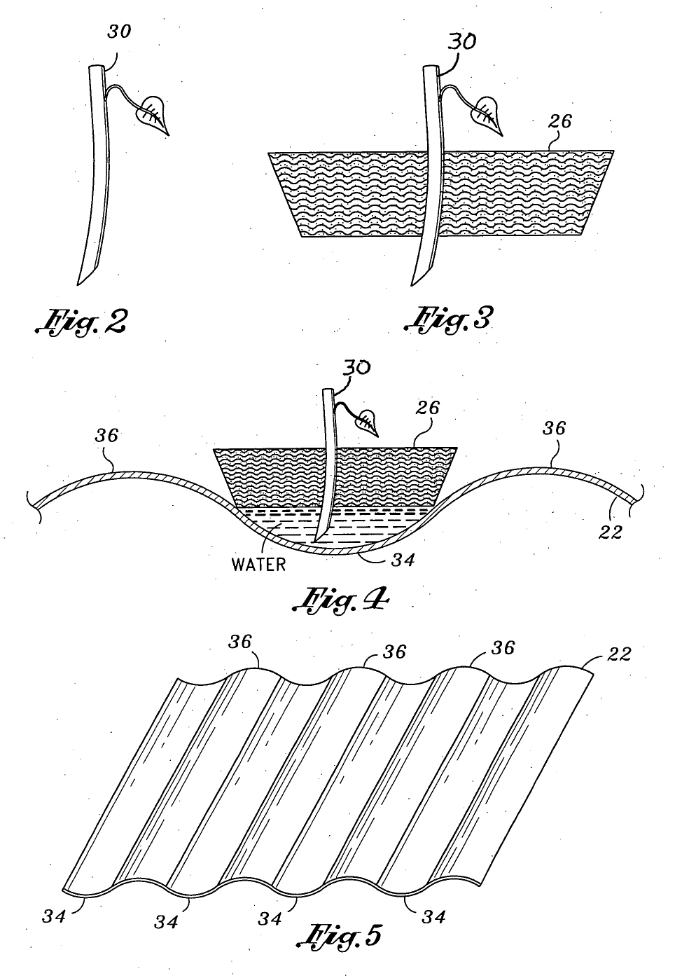

In FIG. 1 a cross-sectional view is also depicted to illustrate the placement of selected plant shoots in porous fibrous plant shoot support medium 26, wherein such porous fibrous plant support medium 26 is placed on the multiple channel extrusion 22. The process of placement of a selected plant shoot is illustrated in FIG. 2 and FIG. 3. A plant shoot 30 is prepared and placed into the porous fibrous plant shoot support medium 26 as illustrated in FIG. 3, with a selected length protruding from the bottom of the porous fibrous plant shoot support medium 26. Such porous plant shoot support medium is then placed within the water and nutrient conducting channel 34 of the multiple channel extrusion 22, as illustrated in FIG. 4. Within such a water and nutrient conducting channel 34, the porous fibrous plant shoot support medium 26 with its plant shoot 30 is repeated with each individual plant shoot 30 and its porous fibrous plant shoot support medium 26 placed end to end along the length of the water and nutrient conducting channel 34 as illustrated in FIG. 1 and FIG. 7. The plant shoot 30 selected length protruding from the bottom of the porous plant shoot support medium 26 is selected to provide for water and nutrient flow over the cut end of the plant shoot 30 as illustrated in FIG. 4 and FIG. 7. The porous plant shoot medium 26 is selected from any porous structure, such as rockwool, which is strong enough to support the plant shoot 30, but loose enough to allow the growing plant shoot 30 to expand without restriction, which will allow for air circulation, and can be easily prepared. The porous plant shoot medium 26 is also selected in a size to be fitted into the water and nutrient conducting channels 34 of the multiple channel extrusion and provide for water and nutrient flow over the end of the plant shoot 30. The porous plant shoot medium 26 density and size is also selected to provide that the emerging root structure of the plant shoot 30 is shielded from growth promoting light 48 to preclude any adverse effects on efficient growth of the root structure of the plant shoot 30. The growth promoting light 48 means may be natural light or may be a selected artificial growth promoting light source.

The multiple channel extrusion 22 illustrated in FIG. 5 can be obtained as any multiple channel extrusion with alternating channels 34 for conducting water and air flow and alternating channel surfaces 36 to divide the water conducting channels 34 from each other. In FIG. 5 the alternating channels 34 are shown as a series of continuous curvilinear surfaces to form the conducting water channels 34 and the separating channels 36. The multiple channel extrusion 22 may consist of rectangular channels or trapezoidal channels; any economical channel form that will receive a plant shoot 30 in its porous fibrous plant shoot support medium 26 and provide for water and nutrient flow over the plant shoot 30 will suffice to provide the function of the multiple channel extrusion 22.

In FIG. 7 the water flow is depicted flowing downward in one of the water conducting channels in the multiple channel extrusion 22. In FIG. 10 in the same cross-section depiction, air flow is depicted flowing upward in the same conducting channel of the multiple channel extrusion 22 with the air flow flowing over the plant shoot root structure 44 that has sprouted at the tip of the plant shoot. The air flow provides for the selected aeration and drying cycle of the plant shoot 30 root structure 44. In FIG. 1 the water and nutrient flow from the multiple channel extrusion 22 is shown as a free fall waterfall 38 back into the water and nutrient supply reservoir 10; such a free fall waterfall 38 causes aeration and stirring of the water and nutrient to occur.

The electrically driven pump 14 depicted in FIG. 1 is shown schematically only and may be any commercially available pump capable of receiving the water and nutrient flow from the reservoir supply duct 18 to the pump 14, which then delivers the water and nutrient flow to the pump supply duct 20. The air supply unit 46 depicted in FIG. 1 is shown schematically only and may be any commercially available fan capable of blowing air over the plant shoots 30 and through the conducting ducts 34 of the multiple channel extrusion 22. The controller 16 depicted in FIG. 1 is shown schematically only and may be any commercially available controller capable of controlling the on-off cycle of the electrically driven pump 14 and the on-off cycle of the air supply unit 46. The on-off cycle of the electrically driven pump 14 and the on-off cycle of the air supply unit 46 are selected for the optimal growth of the root structure 44 of the selected plant shoot 30 that is being grown in the High Efficiency Automatic Plant Cloning device depicted in FIG. 1. The air supply unit 46 may in selected growth cycles be left in the off mode with only natural air flow occurring. The light illumination means 48 depicted in FIG. 1 is for illustration purposes only; such light illumination can be provided by any commercially available source used for enhancing plant growth or even natural sunlight itself.

In practice of the invention, it has been found that the invention is adequately demonstrated by the following. A water and nutrient supply and return reservoir 10 with dimensions of approximate width of 18 inches (approximate 46 cm), approximate length of 27 inches (approximate 69 cm), approximate depth of 5¾ inches (approximate 14.6 cm) made from extrudeable semi-rigid plastic provides such an adequate reservoir. A multiple channel extrusion 22 made from extrudeable plastic with a width of approximately 17¾ inches (approximately 45 cm) and length of 27 inches (approximately 69 cm), a thickness of approximately 1/16 inch (approximately 0.16 cm), and crest to crest distance of approximately 2¾ inches (approximately 7 cm) with channel depth of approximately 9/16 inch (approximately 1.4 cm has provided an adequate multiple channel extrusion 22. For the porous fibrous plant shoot support medium 26 fibrous rockwool in the form of 1 inch by 1 inch by 1 inch (2.54 cm by 2.54 cm by 2.54 cm) cubes have been found to provide adequate support structure for a plant shoot 30. Such porous fibrous pant shoot support medium 26 sizings have been found to provide for adequate air circulation in the drying cycle, while at the same time providing for a loose enough support so as not to impede the growth of the plant shoot 30 and its emerging root structure 44. Such porous fibrous plant shoot support medium 26 sizings have also been found to be sufficient to provide the necessary dark environment for root structure growth 44. It has been further found that such porous fibrous plant shoot support medium 26 sizings provide a combination of air circulation and ultra violet light absorption from the growth light means 48 to maintain a healthy growth environment for the plant shoot 39 and its emerging root structure 44. The porous fibrous plant shoot support medium 26 cubes are located end to end in a water conducting channel 34, and this size provides for an approximate ⅛ inch (0.3 cm) clearance for the water flow down the channel. The angle θ has been used successfully in the 10 to 15 degree range. An electrically driven pump 14 with a capacity of 1.25 gpm (4.8 liters per minute) has successfully provided water and nutrient flow for this configuration. For this configuration, water pump supply duct 20 and reservoir supply duct to pump 18 sizes of nominal ¾ inch (1.9 cm) standard tubing has been successful. A distributing manifold 24 with a nominal tube diameter of ¾ inch (1.9 cm) with a plurality of water distributing orifices 32 with diameters of ⅛ inch (0.3 cm) has been successful. A water and nutrient free fall 38 height of 2 inches (5 cm) has been satisfactory. With this configuration of invention elements, over 100 plant shoots 30 can be successfully propagated from plant shoot 30 to healthy root structure 44, ready for transplanting to soil environments in less than two weeks with a one half hour water immersion cycle two times every eight hours with natural air drying. With directed air drying the root growth cycle can be reduced almost in half. A 500 lumen light source 48 has been found to be adequate for this size of configuration. The invention has been demonstrated with tomatoes, grape vine, roses and strawberries, demonstrating that any plant that can be propagated from a plant shoot can be successfully propagated to root structure with the invention, with the propagated plants having virtually identical characteristics.

In extension of the invention, the structure of FIG. 1 may be replicated in as many units as desired for any selected plant reproduction process. Similarly, the length and width of the apparatus may be selected at any length for the simultaneous growth of selected numbers of plant shoots 30.

These and other variations of the present invention may be practiced by those of ordinary skills in the art, without departing from the spirit and scope of the present invention, which is more particularly set forth in the claims. It will also be understood that although forms of the invention shown and described herein constitute a preferred embodiment of the invention it is not intended to illustrate all the possible forms of the invention. The words used are words of description rather than limitation. Various changes and variations may be made to the present invention without departing from the spirit and scope of the following claims.

1. A system for growing plant shoots without soil to rooting status, which system comprises:
a water and nutrient supply and return reservoir container adapted for receiving and supplying water and nutrient;
a reservoir supply duct for supply of water and nutrient to a pump means;
an electrically driven pump means for providing circulation of water and nutrient;
an electronic controller means for controlling the on-off cycle of a pump means and air supply means;
a pump supply duct for supplying water and nutrient from the pump means to a distributing manifold;
a distributing manifold to receive water and nutrient from a pump supply duct;
a distributing manifold with a plurality of water and nutrient supply orifices to distribute water and nutrient to a multiple channel extrusion means;
a multiple channel extrusion means with alternating channels to receive water and nutrient for supply to plant shoot material and discharge of water and nutrient into a waterfall into a water and nutrient supply and return reservoir;
a porous fibrous plant support medium for supporting plant shoot materials without plant shoot growth restriction in water and nutrient conducting channels of a multiple channel means and which further provides a darkened environment for root structure growth;
a supporting porous plate means to provide support of a multiple channel means;
a support means to maintain an angle θ between a multiple channel means and a supporting porous plate means;
an air supply means;
a growth light illumination means.

2. A system for growing plant shoots without soil to rooting status of claim 1 wherein no automated air supply means is provided and he system utilizes only natural air diying when the water and nutrient supply by the pump means is turned off by the electronic controller.

3. A system for growing plant shoots without soil to roofing status of claim 1 wherein the multiple channel extrusion means consists of repeating curvilinear surfaces which form alternating water and nutrient conducting channels separated by non conducting surfaces.

4. A system for growing plant shoots without soil to rooting status of claim 1 wherein the supporting porous plate means consists of a rectangular plate with large boles separated by ribs in the supporting porous plate to provide structural strength.

5. A system for growing plant shoots without soil to rooting status of claim 1, wherein the multiple channel extrusion means consists of repeating and alternating rectangular channels which form alternating water and nutrient conducting channels separated by non conducting surfaces.

6. A system for growing plant shoots without soil to rooting status of claim 1 wherein the multiple channel extrusion means consists of repeating and alternating trapezoidal channels which form alternating water and nutrient conducting channels separated by non conducting surfaces.

7. A system for growing plant shoots without soil to rooting status of claim 1, wherein the artificial growth light source is eliminated in favor of natural light.

8. A method for growing plant shoots without soil to rooting status comprising the steps of:
providing an open water and nutrient supply return reservoir for water and nutrient;
providing a reservoir supply duct disposed to receive water and nutrient from the water and nutrient supply return reservoir;
providing an electrically driven pump disposed to receive water and nutrient from the reservoir supply duct;
providing an electronic controller for controlling the on-off cycles of pump water and nutrient and air supply,
providing a pump supply duct disposed to receive water and nutrient from the electrically driven pump;
providing a distributing manifold with a plurality of water and nutrient orifices to receive and distribute water and nutrient through such orifices, such distributing manifold disposed to receive water and nutrient from the pump supply duct;
providing a multiple channel extrusion further comprising equally spaced and alternating water and nutrient conducting channels separated by non conducting surfaces with such water and nutrient conducting channels disposed to receive water and nutrient flow from the water and nutrient plurality of water and nutrient supply orifices in the distributing manifold and discharge such water and nutrient through a waterfall into a water and nutrient supply and return reservoir to create aeration and stirring of water and nutrient;
providing a porous supporting plate disposed to provide a surface to support the multiple channel extrusion;
providing a support to maintain an angle θ between the multiple channel extrusion and the supporting porous plate;
providing a porous fibrous support medium for supporting plant shoots and not impeding plant shoot growth while providing a darkened environment for plant shoot root structure growth such porous fibrous support medium disposed with placement in the water and nutrient conducting channel of the multiple channel extrusion;
providing an air circulation method disposed to provide air circulation;
providing a plant growth enhancement light method disposed to provide light to the plant shoots.
